(19) United States
(12) Patent Application Publication
(10) Pub. No.: US 2009/0142380 A2
(43) Pub. Date: Jun. 4, 2009

(54) COSMETIC COMPOSITION IN POWDERED FORM

(75) Inventors: Nathalie GEFFROY, Paris (FR); Audrey VALOIS, Villiere le Bacie (FR)

Correspondence Address: FINNEGAN, HENDERSON, FARABOW, GARRETT & DUNNER;LLP, 901 NEW YORK AVENUE, NW, WASHINGTON, DC, 20001-4413, UNITED STATES, 202-408-4000

(73) Assignee: L'Oreal, Paris (FR)

(21) Appl. No.: 11/826,649

(22) Filed: Jul. 17, 2007

(51) Int. Cl. A61K 8/02; A61Q 1/02

(52) U.S. Cl. 424/401; 514/769; 424/63

(57) ABSTRACT

Disclosed herein is an anhydrous cosmetic composition in powdered form comprising at least one pulverulent phase and at least one fatty binder, the said pulverulent phase comprising i) 30 to 70% by weight, relative to the total weight of the composition, of at least one pigment, and ii) 30 to 70% by weight, relative to the total weight of the composition, of at least one lamellar particle. Disclosed herein is also a method for making up the skin, comprising applying the composition to the skin, and a method for concealing the color defects of the skin, while conferring a natural and satiny appearance on the made-up skin.

This application claims benefit of U.S. Provisional Application No. 60/831,445, filed Jul. 18, 2006, the contents of which are incorporated herein by reference.

Disclosed herein is a cosmetic make-up composition for the skin in powdered form, comprising a high content of pigments. Also disclosed herein is a method for making up the skin comprising applying the composition to the skin.

The make-up composition according to the present disclosure is, in at least one embodiment, a make-up composition for the skin in powdered form, such as a face or body powder, an eye shadow, a blusher or a concealer product. In at least one embodiment, the presently disclosed composition is a face powder.

Make-up compositions for the skin are commonly used to give an aesthetic color to the skin, such as to the face, but also to conceal skin imperfections such as blotches and spots.

Some make-up compositions are provided in loose powder or compact powder form. These compositions generally comprise a high content of powders, such as at least 80% by weight, relative to the total weight of the composition.

Among the powders commonly used, zinc oxide or titanium dioxide are of interest for providing covering power (that is to say allowing good concealment of skin color defects) and also make it possible to protect the skin from the ultraviolet rays of the sun.

However, when they are introduced into cosmetic formulas, these powders may have the disadvantage of conferring onto the products a dry and rough feel during their application to the skin and thus hampering the obtaining of properties of softness.

In addition, when these powders are applied to the skin, the make-up obtained is often very opaque, visible, and has a pronounced powdery appearance which is not close to the natural grain of the skin: such a make-up is not natural-looking.

The compositions in powdered form are often preferred by users having a skin with a tendency to be greasy because of their texture. However, the make-up obtained by loose powders has a poor resistance over time to rubbing or to sebum.

It would be desirable, therefore, to have available a make-up composition for the skin in powdered form having good spreading properties during its application to the skin and conferring a make-up capable of effectively concealing skin color defects with no “mask” effect. Indeed, when the compositions in powdered form exhibit good slip, the user can work it more easily when they apply it to the skin, and the make-up obtained is more homogeneous.

The present inventors have found that by combining specific particles in a composition in powdered form, it is possible to obtain a make-up which makes it possible to effectively conceal skin defects, such as color defects, while conferring a natural and satiny appearance on the made-up skin. The compositions obtained also may exhibit good properties of softness during their application to the skin.

Thus, disclosed herein is a cosmetic composition in powdered form comprising at least one pulverulent phase and at least one fatty binder, the pulverulent phase comprising:

i) 15 to 70% by weight, relative to the total weight of the composition, of at least one pigment, and,

ii) 30 to 85% by weight, relative to the total weight of the composition, of at least one lamellar particle other than the pigment.

Disclosed herein is also a method for making up the skin of the face and/or of the body, comprising applying to the skin a composition as defined above.

Further disclosed herein is a method of concealing skin color defects, while conferring a natural and satiny appearance on the made-up skin.

The composition disclosed herein is provided in powdered form which may be, for example, a loose powder or a compact powder (which can be disintegrated in the dry state or with water).

Pulverulent Phase

The composition disclosed herein comprises at least one pulverulent phase comprising at least one pigment and at least one lamellar particle.

Pigments

The expression “pigments,” as used herein, means particles of any shape, which are white or colored, inorganic or organic, insoluble in the physiological medium and intended to color the composition. Thus, the pigments used herein may be white and/or colored, inorganic and/or organic.

According to at least one embodiment, the composition disclosed herein may comprise at least one pigment chosen from inorganic pigments. These inorganic pigments may, for example, be chosen from pigments of metal oxides.

In at least one embodiment, the inorganic pigments may be chosen from, for example, titanium dioxide, which is optionally surface-treated, zirconium or cerium oxides and zinc, iron (black, yellow or red) or chromium oxides, manganese violet, ultramarine blue, chromium hydrate and ferric blue, metallic powders such as aluminium powder and copper powder.

According to at least one embodiment, the inorganic pigments, such as metal oxides, present in the composition disclosed herein are chosen from titanium dioxide, zinc oxide and/or iron oxide.

According to another embodiment, the pulverulent phase of the composition according to the present disclosure may comprise at least two different pigments.

According to a further embodiment, the pulverulent phase of the composition disclosed herein may comprise at least a first pigment chosen from iron oxides and at least a second pigment chosen from zinc oxides and/or titanium dioxide.

The iron oxides may be present in the composition according to the present disclosure in an amount ranging from 1 to 40% by weight, relative to the total weight of the composition, such as from 3 to 30%, or further from 4 to 25%, by weight.

The zinc oxides may be present in the presently disclosed composition in an amount ranging from 5 to 55% by weight, relative to the total weight of the composition, such as from 10 to 40% by weight, or further from 15 to 25% by weight.

The titanium dioxide may be present in the presently disclosed composition in an amount ranging from 3 to 55% by weight, relative to the total weight of the composition, such as from 10 to 40% by weight, or further from 15 to 25% by weight.

According to one embodiment, the zinc oxide and titanium dioxide pigments may be present in the composition in an amount such that the zinc oxide to titanium dioxide weight ratio ranges from 0.5 to 2, such as from 0.8 to 1.8, or further from 0.9 to 1.7.

According to another embodiment, the iron oxide and zinc oxide pigments and the titanium dioxide pigments may be present in the composition in an amount such that the weight ratio of the sum of the zinc oxides and titanium dioxides to the iron oxides ranges from 0.1 to 10, such as from 0.2 to 8.

In addition to the inorganic pigments, the pulverulent phase of the composition disclosed herein may comprise organic pigments. Among the organic pigments, there may be mentioned, for example, carbon black, D & C type pigments, lacquers based on carmine, barium, strontium, calcium and aluminum.

The pulverulent phase may also comprise goniochromatic pigments; these pigments may exhibit a relatively high change of color with the angle of observation.

The goniochromatic pigment may be chosen, for example, from pigments with interferential multilayer structure and liquid crystal pigments.

In the case of a multilayer structure, the latter may comprise, for example, at least two layers, each layer, independently of the other layer(s) or not, being produced, for example, from at least one material chosen from the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and combinations thereof. The goniochromatic pigments with interferential multilayer structures according to the present disclosure may be chosen from, for example, those described in the following documents: U.S. Pat. No. 3,438,796, EP-A-227 423, U.S. Pat. No. 5,135,812, EP-A-1 70 439, EP-A-341 002, U.S. Pat. No. 4,930,866, U.S. Pat. No. 5,641,719, EP-A-472 371, EP-A-395 410, EP-A-753 545, EP-A-768 343, EP-A-571 836, EP-A-708 154, EP-A-579 091, U.S. Pat. No. 5,411,586, U.S. Pat. No. 5,364,467, WO-A-97/39066, DE-A-42 25 031, WO 95/17479 (BASF), DE-A-196 14 637. They may exist in the form of glitter with a metallic color.

The goniochromatic pigments with an interferential multilayer structure may be chosen, for example, from the following commercial goniochromatic pigments: INFINITE COLORS from the company Sihiseido, SICOPEARL FANTASTICO from BASF, COLORSTREAM, XIRALLIC, XIRONA from Merck, COLORGLITTER from FLEX, and mixtures thereof.

The multilayer structures that can be used in the invention include, for example, the following structures: Al/SiO₂/Al/SiO₂/Al; Cr/MgF₂/Al/MgF₂/Al; MoS₂/SiO₂/Al/SiO₂/MoS₂; Fe₂O₃/SiO₂/Al/SiO₂/Fe₂O₃; Fe₂O₃/SiO₂/Fe₂O₃/SiO₂/Fe₂O₃; MoS₂/SiO₂/mica-oxide/SiO₂/MoS₂; Fe₂O₃/SiO₂/mica-oxide/SiO₂/Fe₂O₃. Various colors may be obtained according to the thickness of the various layers. Thus, with the Fe₂O₃/SiO₂/Al/SiO₂/Fe₂O₃ structure, the color goes from golden-green to grey-red for SiO₂ layers from 320 to 350 nm; from red to golden for SiO₂ layers from 380 to 400 nm; from violet to green for SiO₂ layers from 410 to 420 nm; from copper to red for SiO₂ layers from 430 to 440 nm.

Consequently, the multilayer structure may be mainly inorganic or organic. As noted, various colors may be obtained depending on the thickness of each of the various layers.

Liquid crystal pigments are, for example, described in application EP-A-1 046 692.

As liquid crystal particles, it is possible to use, for instance, those known under the CTFA name polyacrylate-4 and sold under the names “HELICONE® HC Sapphire”, “HELICONE® HC Scarabeus”, “HELICONE® HC Jade”, “HELICONE® HC Maple”, “HELICONE® HC XL Sapphire”, “HELICONE® HC XL Scarabeus”, “HELICONE® HC XL Jade”, “HELICONE® HC XL Maple” by the company WACKER.

The pigments may be present in the composition of the present disclosure in an amount ranging from 15 to 70% by weight, relative to the total weight of the composition, such as from 25 to 70% by weight, or further from 26 to 60% by weight, such as from 27 to 50% by weight.

Lamellar Particles

The pulverulent phase of the composition disclosed herein comprises at least one lamellar particle other than the pigments described above.

The expression “lamellar particle,” for the purposes of the present disclosure, means particles existing in the form of optionally stratified sheets.

These sheets may be characterized by a thickness which is smaller than the largest dimension. In at least one embodiment, the ratio between the largest dimension and the thickness may range from 2 to 100.

The lamellar particles present in the composition of the present disclosure may be chosen from lamellar pearlescent agents, lamellar fillers and/or lamellar reflecting particles.

The expression “pearlescent agents,” as used herein, means iridescent particles, such as those produced by certain mollusks in their shell or alternatively synthesized.

The lamellar pearlescent agents may be chosen from white pearlescent agents such as mica coated with titanium or with bismuth oxychloride, colored pearlescent agents such as mica-titanium coated with iron oxides, mica-titanium coated with, for example, ferric blue or chromium oxide, mica-titanium coated with an organic pigment of the abovementioned type and pearlescent agents made of bismuth oxychloride or based on bismuth oxychloride.

According to at least one embodiment, the lamellar pearlescent agent is a pearlescent agent made of bismuth oxychloride, such as that sold under the name BIRON LF 2000 by the company MERCK.

The lamellar pearlescent agents may be present in the composition disclosed herein in an amount ranging from 3 to 70% by weight, relative to the total weight of the composition, such as 5 to 40% by weight.

The lamellar fillers may be chosen from:

talc which is a magnesium silicate hydrate, for example those marketed under the names “Talc Luzenac 00” by the company LUZENAC and “Talc P3” by the company NIPPON TALC;

kaolin which may be an aluminium silicate hydrate that exists in the form of particles with an anistropic shape having sizes generally less than 30 μm; as kaolin, it is possible to use, for example, the one sold under the name “Kaolin Supreme 1” from ENGLISH CHINA CLAYS;

boron nitride, such as those marketed under the names “Ceram Blanche 1”, “Ceram Blanche” sold by the company SPCI, and “PUHP 3008” sold by the company Saint Gobain Ceramics;

mica, or aluminosilicate, which may be chosen from muscovite, phlogopite, tiotite, sericite, lepidolite, paragonite, margarite, roscoelite, artificial or synthetic mica having a fluorine atom replacing the hydroxyl group of the natural mica, and the baked or calcined products of these micas. Micas generally exist in the form of scales having dimensions of 2 to 200 μm, such as 5-70 μm, and a thickness of 0.1 to 5 μm, such as 0.2-3 μm. As mica, it is possible, for example, to use those sold under the names “MICA SFG70” by the company ASPANGER and “MICA CONCORD 1000” by the company SCIAMA;

lamellar silica, such as that sold under the names “SG Flake 3 M” by the company MAPRECOS and “Chemicelen” by the company SUMITOMO;

and mixtures thereof.

According to at least one embodiment, the lamellar fillers may be chosen from talc, mica, boron nitride, kaolin and mixtures thereof.

According to at least one embodiment, talc may be present in the composition disclosed herein in an amount ranging from 5 to 55% by weight, relative to the total weight of the composition, such as from 10 to 53% by weight.

According to at least one embodiment, boron nitride may be present in the composition disclosed herein in an amount ranging from 3 to 10% by weight, relative to the total weight of the composition, such as from 5 to 8% by weight.

The lamellar fillers may be present in the composition disclosed herein in an amount ranging from 20 to 70% by weight, such as from 25% to 50% by weight, or further from 30% to 35% by weight.

The lamellar particles may be chosen from lamellar reflecting particles.

The expression “reflecting particles,” as used herein, means particles whose size, structure, such as the thickness of the layer(s) constituting it and their physical and chemical nature, and the surface state, allow them to reflect the incident light. This reflection may, where appropriate, have a sufficient intensity to create at the surface of the composition or of the mixture, when it is applied to the support to be made up, super bright spots which are visible to the naked eye, that is to say more luminous spots which contrast with their environment by appearing to shine.

The lamellar reflecting particles may or may not have a multilayer structure and, in the case of a multilayer structure, for example at least one layer with a uniform thickness, such as a reflecting material.

When the reflecting particles have a multilayer structure, they may, for example, comprise a natural or synthetic substrate, such as a synthetic substrate that is at least partially coated with at least one layer of a reflecting material, for instance at least one metal or metallic material. This substrate may be monomaterial, multimaterial, organic and/or inorganic. For example, it may be chosen from glass, ceramics, graphite, metal oxides, alumina, silicas, silicates such as aluminosilicates and borosilicates, synthetic mica, and mixtures thereof, this list not being limiting.

The reflecting material may comprise a layer of metal or of a metallic material.

Reflecting particles are described, for example, in the documents JP-A-09-188830, JP-A-10-158450, JP-A-10-158541, JP-A-07-258460, and JP-A-05-017710.

As an example of reflecting particles comprising an inorganic substrate coated with a metal layer, there may also be mentioned particles comprising a borosilicate substrate coated with silver.

Particles containing a glass substrate coated with silver, in the form of platelets, are sold under the name MICROGLASS METASHINE REFSX 2025 PS by the company TOYAL. Particles containing a glass substrate coated with a nickel/chromium/molybdenum alloy are sold under the name CRYSTAL STAR GF 550, GF 2525 by the same company.

The lamellar reflecting particles may also be chosen from particles containing a synthetic substrate that is at least partially coated with at least one layer of at least one metallic material, such as a metal oxide, chosen for example from titanium oxides such as TiO₂, iron oxides such as Fe₂O₃, tin oxides, chromium oxides, barium sulphate and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂ and mixtures or alloys thereof.

By way of example of such particles, there may be mentioned, for example, particles comprising a synthetic mica substrate coated with titanium dioxide, or particles of glass coated either with brown iron oxide, titanium oxide, tin oxide or mixtures thereof such as those sold under the trademark REFLECKS® by the company ENGELHARD.

The reflecting particle(s) may be present in the composition disclosed herein in an amount ranging from 5 to 70% relative to the total weight of the first composition, such as from 10% to 50% by weight, or further from 20 to 40% by weight.

According to at least one embodiment, the pulverulent phase of the composition disclosed herein may comprise at least two lamellar particles of a different nature.

According to another embodiment, the pulverulent phase of the composition disclosed herein may comprise at least two lamellar particles of a different nature, one of the lamellar particles being chosen from lamellar pearlescent agents, the other lamellar particle being chosen from lamellar fillers.

According to a further embodiment, the pulverulent phase of the composition disclosed herein may comprise at least one lamellar pearlescent agent and at least two lamellar fillers.

The lamellar particle(s) may be present in the pulverulent phase in an amount ranging from 30 to 85% by weight, relative to the total weight of the composition, such as from 40 to 75% by weight, or further from 40 to 70% by weight.

According to at least one embodiment, the lamellar particles and the pigments are present in the composition in an amount such that the weight ratio of the lamellar particles to the pigments ranges from 0.5 to 5, such as from 0.8 to 2.5.

Additional Particles

In addition to the lamellar particles described above, the composition disclosed herein may comprise at least one additional non-lamellar particle of any shape, for example of spherical or oblong shape.

There may be mentioned spherical silica, polyamide (Nylon®) powders, poly-β-alanine powders, polyethylene powders, polyurethane powders such as the hexamethylene diisocyanate and trimethylol hexyl lactone copolymer powder sold under the names PLASTIC POWDER D-400 by the company TOSHIKI, powders of tetrafluoroethylene polymers (Teflon®), lauroyllysine, starch, boron nitride, polymeric hollow microspheres such as those of polyvinylidene chloride/acrylonitrile such as Expancel® (Nobel Industrie), acrylic acid copolymers, silicone resin powders, for example silsesquioxane powders (silicone resin powders such as those described in patent EP 293 795; Tospearls® from Toshiba, for example), particles of elastomeric polyorganosiloxanes, particles of polymethyl methacrylate, precipitated calcium carbonate, magnesium carbonate and hydrocarbonate, hydroxyapatite, hollow silica microspheres, glass or ceramic microcapsules, metallic soaps derived from organic carboxylic acids having from 8 to 22 carbon atoms, such as 12 to 18 carbon atoms, and for example zinc, magnesium or lithium stearate, zinc laurate, magnesium myristate, barium sulphate and mixtures thereof.

Fatty Binder

The composition disclosed herein comprises at least one fatty binder.

The expression “fatty binder,” as used herein, means a fatty phase which generally comprises at least one oil. This type of fatty phase serves, for instance, as dispersing medium for the pulverulent phase.

The fatty binder may comprise at least one oil.

The oil may be chosen from the oils conventionally used as binder in loose or compact powders. For example, these oils may be chosen from:
mink oil, turtle oil, soybean oil, grapeseed oil, sesame oil, corn oil, rapeseed oil, sunflower oil, cottonseed oil, avocado oil, olive oil, castor oil, jojoba oil, peanut oil;
hydrocarbon oils, such as paraffin oils, squalane, petroleum jelly;
fatty esters, such as isopropyl myristate, isopropyl palmitate, butyl stearate, isodecyl stearate, isocetyl stearate, hexyl laurate, isononyl isononanoate, 2-ethylhexyl palmitate, 2-hexyldecyl laurate, 2-octyldecyl palmitate, 2-octyldodecyl myristate or lactate, 2-diethylhexyl succinate, diisostearyl malate, glycerine or diglycerine triisostearate;

silicone oils such as polymethylsiloxanes, polymethylphenylsiloxanes, polysiloxanes modified by fatty acids, fatty alcohols or polyoxyalkylenes, fluorinated silicones or perfluorinated oils;

higher fatty acids such as myristic acid, palmitic acid, stearic acid, behenic acid, oleic acid, linoleic acid, linolenic acid or isostearic acid;

higher fatty alcohols such as cetanol, stearyl alcohol or oleyl alcohol;

polymethylfluoroalkyl dimethylsiloxanes of formula (I): 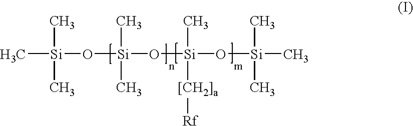
in which:

n is an integer ranging from 5 to 90, such as from 30 to 80, or from 50 to 80,

m is an integer ranging from 1 to 150, such as from 1 to 80, or from 1 to 40,

a is an integer ranging from 0 to 5, and

Rf is a perfluoroalkyl radical comprising from 1 to 8 carbon atoms; and

mixtures thereof.

According to at least one embodiment, the oil may be chosen from silicone oils such as polymethylsiloxanes, polymethylphenylsiloxanes, polysiloxanes modified with fatty acids, fatty alcohols or polyoxyalkylenes, fluorinated silicones or perfluorinated oils such as isopropyl myristate, isopropyl palmitate, butyl stearate, isodecyl stearate, isocetyl stearate, hexyl laurate, isononyl isononanoate, 2-ethylhexyl palmitate, 2-hexyldecyl laurate, 2-octyldecyl palmitate, 2-octyldodecyl myristate or lactate, 2-diethylhexyl succinate, diisostearyl malate, glycerine or diglycerine triisostearate.

The silicone oil may be chosen from linear silicone oils of low viscosity (ranging from 1 to 300 cSt).

In at least one embodiment, the silicone oil may be linear polysiloxane comprising (except terminal groups) units of formula (I) 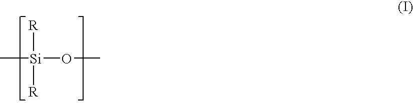
in which each substituent R independently is a lower alkyl group (having 1 to 6 carbon atoms).

The degree of polymerization (number of repeating units) of these polysiloxanes of low viscosity may range, for example, from 3 to 2000.

These silicone oils of low viscosity may be prepared according to known methods, or bought from the market: there may be mentioned, for example, Silbione series 47 oil (RHONE POULENC), series 200 oil (DOW CORNING), SF 96 oil (GENERAL ELECTRIC) and mixtures thereof.

The terminal groups may be, for example, trimethylsilyl, dimethyl hydroxymethylsilyl or vinyl dimethylsilyl groups.

The silicone oil may range from 12 to 98.9% by weight relative to the total weight of the silicone binder.

The fatty ester oil may be isononyl isononanoate.

The oil may be present in the composition disclosed herein in an amount ranging from 0.5 to 15% by weight, relative to the total weight of the composition, such as from 1 to 10% by weight, or further from 1.5 to 5% by weight.

In addition to the oil, the fatty binder of the composition disclosed herein may comprise a wax and/or silicone pasty fatty substances.

The waxes (and/or silicone pasty fatty substances) which can be used in the fatty binder of the composition disclosed herein are substituted polysiloxanes, such as those of low melting point. They may be substituted linear polysiloxanes mainly comprising (apart from the terminal groups) units of formulae II and III, in the respective molar proportions m and n: 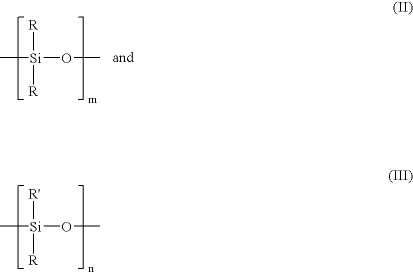
in which:

each substituent R is as defined above,

each R′ independently is an optionally unsaturated (linear or branched) alkyl having 6-30 carbon atoms, or alternatively a group —X—R″, each X independently being:
—O—,
—(CH2)a-O—CO—,
—(CH2)b-CO—O—,
a and b independently are numbers that may range from 0 to 6, and each R″ independently is an optionally unsaturated alkyl group having 6 to 30 carbon atoms,

m is a number that may range from 0 to 400, such as from 0 to 100,

n is a number that may range from 1 to 200, such as from 1 to 100, the sum (m+n) being less than 400, such as less than or equal to 100.

These silicone waxes are known or may be prepared according to known methods. Among the commercial silicone waxes of this type, there may be mentioned for example those sold under the names Abilwax 9800, 9801 or 9810 (GOLDSCHMIDT), KF910 and KF7002 (SHIN ETSU), or 176-1118-3 and 176-11481 (GENERAL ELECTRIC).

The silicone waxes which can be used may also be chosen from the compounds of formula (IV):
R₁—Si(CH₃)₂—O—[Si(R)₂—O—]_{z}—Si(CH₃)₂—R₂ (IV)
in which:

R is as defined above,

R₁ is an alkyl group having from 1 to 30 carbon atoms, an alkoxy group having from 6 to 30 carbon atoms, or a group of formula:

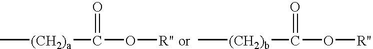

R₂ is an alkyl group of 6 to 30 carbons atoms, an alkoxy group having from 6 to 30 carbon atoms or a group of formula: 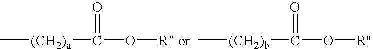
a and b is a number from 0 to 6,

R″ is an alkyl having from 6 to 30 carbon atoms, and z is a number which may range from 1 to 100.

Among the silicone waxes of formula (IV), which are known products or products which may be prepared according to known methods, there may be mentioned for example the following commercial products: Abilwax 2428, 2434 and 2440 (GOLDSCHMIDT), or VP 1622 and VP 1621 (WACKER).

The wax and/or the silicone pasty fatty substance may range from 1 to 60% by weight relative to the total weight of the silicone binder.

In addition to the oil and/or the silicone wax, the fatty binder of the composition disclosed herein may comprise a silicone resin.

Silicone resins are products of hydrolysis or of polycondensation of mixtures of siloxanes of formulae (R)₃SiOCH₃ and Si(OCH₃)₄, R being an alkyl group having from 1 to 6 carbon atoms.

These silicone resins are known or may be prepared according to known methods. Among the commercial silicone resins which can be used, there may be mentioned for example those sold under the names DC 593 (DOW CORNING) or SS 4230 (GENERAL ELECTRIC).

The silicone resin may be present in an amount ranging from 0.1 to 25% by weight relative to the total weight of the silicone binder.

According to one embodiment, the fatty binder of the composition disclosed herein comprises at least two components chosen from:

(a) a silicone oil,

(b) a wax and/or a silicone pasty fatty substance,

(c) a silicone resin.

The fatty binder may be present in the composition in an amount ranging from 1 to 8% by weight, relative to the total weight of the composition, such as from 2 to 7% by weight, further from 3 to 6% by weight, or still further from 2 to 5% by weight.

Additional Wax

In addition to the wax which may be present in the fatty binder, the composition disclosed herein may comprise at least one additional wax.

The expression “wax”, for the purposes of the present disclosure, means a lipophilic fatty compound which is solid at room temperature (25° C.) and atmospheric pressure, (760 mmHg, that is 10⁵ Pa), which exhibits a reversible solid/liquid change of state, which may have for example a melting point greater than or equal to 30° C., such as greater than or equal to 55° C., and which may be up to 250° C., such as up to 230° C., and in at least one embodiment, up to 120° C.

By heating the wax to its melting point, it is possible to make it miscible with oils and to form a microscopically homogeneous mixture, but on allowing the temperature of the mixture to return to room temperature, recrystallization of the wax in the oils of the mixture is obtained.

The melting point values correspond, for the purposes of the present disclosure, to the melting peak measured with the aid of differential scanning calorimetry (D.S.C.), for example the calorimeter sold under the name DSC 30 by the company METLER, with a temperature rise of 5 or 10° C. per minute.

The waxes, for the purposes of the present disclosure, may be those generally used in the cosmetic or dermatological fields. They may optionally be hydrocarbon-based, silicone-based and/or fluorinated, optionally comprising ester or hydroxyl functional groups. They may also be of natural or synthetic origin. By way of non-limiting illustration of these waxes, there may be mentioned, for example:

beeswax, lanolin wax, and Chinese waxes; rice wax, Carnauba wax, Candelilla wax, Ouricury wax, cork fibre wax, sugar cane wax, Japan wax and sumac wax; montan wax, microcrystalline waxes, paraffin waxes, ozokerites, ceresin wax, lignite wax, polyethylene waxes, the waxes obtained by Fisher-Tropsch synthesis, fatty acid esters and glycerides which are concrete at 40° C., such as at more than 55° C.,

waxes obtained by catalytic hydrogenation of animal or vegetable oils having linear or branched C₈-C₃₂ fatty chains, such as hydrogenated jojoba oil, hydrogenated sunflower oil, hydrogenated castor oil, hydrogenated copra oil and hydrogenated lanolin oil,

silicone waxes or fluorinated waxes, and

mixtures thereof.

According to at least one embodiment, the solid fatty phase may comprise at least one wax chosen from carnauba waxes, paraffin waxes and mixtures thereof.

According to another embodiment, the additional wax present in the composition disclosed herein may be totally or partially in powdered, such as micronized, form in order to facilitate its use in the preparation of the cosmetic composition.

Among the waxes which can be used in powdered form, there may be mentioned, for example, the Carnauba wax microbeads sold under the name Microcare 350® by the company Micro Powders and paraffin wax microbeads sold under the name Microease 114S® by the company Micro Powders. Such additional micronized waxes may make it possible to improve the properties during the application of the composition to the skin.

The additional waxes may be present in the composition disclosed herein in an amount ranging from 0.5 to 15% by weight, relative to the total weight of the composition, such as from 0.8 to 5%, or further from 0.9 to 1.2% by weight.

Additives

The composition according to the present disclosure may comprise at least one other customary cosmetic ingredient which may be chosen, for example, from lipophilic gelling and/or thickening agents, antioxidants, perfumes, preservatives, neutralizing agents, sunscreens, vitamins, moisturizers, self-tanning compounds, antiwrinkle actives, emollients, hydrophilic or lipophilic actives, anti-pollution or anti-free radical agents, sequestrants, film-forming agents, non-elastomeric surfactants, skin-relaxing actives, soothing agents, agents stimulating the synthesis of skin or epidermal macromolecules and/or preventing their degradation, anti-glycation agents, anti-irritant agents, desquamating agents, depigmenting agents, anti-pigmenting agents or pro-pigmenting agents, NO-synthase inhibitors, agents stimulating the proliferation of fibroblasts or keranocytes and/or the differentiation of keranocytes, agents acting on the microcirculation, agents acting on the energy metabolism of the cells, wound-healing agents, and mixtures thereof.

According to one embodiment, the composition disclosed herein may be a cosmetic composition in powdered form, comprising:

i) 1 to 10% by weight, relative to the total weight of the composition, of at least one pigment chosen from iron oxides,

ii) 20 to 50% by weight, relative to the total weight of the composition, of pigments chosen from zinc oxides, titanium oxides and a mixture thereof,

iii) 25 to 45% by weight, relative to the total weight of the composition, of at least one lamellar pearlescent agent,

iv) 15 to 35% by weight, relative to the total weight of the composition, of at least one lamellar filler,

v) 2 to 7% by weight, relative to the total weight of the composition, of at least one fatty binder.

According to at least one embodiment, the composition disclosed herein may be an anhydrous composition. The expression “anhydrous composition,” as used herein, means a composition containing less than 2% by weight of water, or even less than 0.5% of water, and in at least one embodiment free of water, water not being added during the preparation of the composition but corresponding to the residual water provided by the ingredients mixed.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below.

### EXAMPLES

### Examples 1 and 2

### Loose Powders

Two loose powders having the following compositions were prepared:

| | | Ex. 1 | Ex. 2 | Ex. 3 |
|---|---|---|---|---|
| | MP Name | (% by weight) | (% by weight) | (% by weight) |
| Phase | Yellow iron | 4.0 | 13.4 | 6.00 |
| A1 | oxide | | | |
| | Red iron oxide | 1.3 | 7.6 | 1.7 |
| | Black ion oxide | 0.4 | 4.0 | 0.7 |
| | Bismuth oxy- | 30.0 | 5.0 | 36.5 |
| | chloride | | | |
| | Titanium di- | 20.0 | 3.0 | 20.0 |
| | oxide | | | |
| | Zinc oxide | 20.0 | 5.0 | — |
| | Boron nitride | 6.0 | 6.0 | 6.0 |
| | Carnauba wax | 1.0 | 1.0 | 1.0 |
| | Mica | | | 20.9 |
| | Talc | 13.7 | 51.4 | — |
| Phase | Silicone binder | 3.0 | 3.0 | |
| A2 | Hydrocarbon | | | 2.00 |
| | binder | | | |
| | Vitamin B5 | | | qs |
| | Aloe extract | | | qs |
| | Vitamin E | | | qs |
| | Preservative | 0.6 | 0.6 | |

All the constituents of phase A1 were mixed in a Lodige mixer for 15 minutes (without emptying).

The constituents of phase A2 were added, with stirring, to A1 and the mixture was mixed for 15 minutes.

The mixture obtained was then ground in a pin mill at a speed of 1800 revolutions/min.

The mixture obtained was sieved on a 400 micron sieve.

The loose powder obtained had good properties of softness during its application to the skin. After application, the make-up made it possible to effectively conceal the color defects of the skin, while conferring a natural and satiny appearance on the made-up skin.

What is claimed is:

1. An anhydrous cosmetic composition in powdered form comprising at least one pulverulent phase and at least one fatty binder, said pulverulent phase comprising:
i) 15 to 70% by weight, relative to the total weight of the composition, of at least one pigment, and,
ii) 30 to 85% by weight, relative to the total weight of the composition, of at least one lamellar particle other than the at least one pigment.

2. A composition according to claim 1, wherein the at least one pigment is chosen from pigments of metal oxides.

3. A composition according to claim 2, wherein the pigments of metal oxides are chosen from pigments of iron oxide, zinc oxide and titanium dioxide.

4. A composition according to claim 1, wherein the at least one pulverulent phase comprises at least two different pigments.

5. A composition according to claim 4, wherein the at least one pulverulent phase comprises at least a first pigment chosen from pigments of iron oxide and at least a second pigment chosen from pigments of zinc oxide and titanium dioxide.

6. A composition according to claim 6, wherein the pigments are present in the composition in an amount ranging from 27 to 50% by weight, relative to the total weight of the composition.

7. A composition according to claim 5, wherein the pigments of iron oxide are present in the composition in an amount ranging from 1 to 40% by weight, relative to the total weight of the composition.

8. A composition according to claim 7, wherein the pigments of iron oxide are present in the composition in an amount ranging from 4 to 25% by weight, relative to the total weight of the composition.

9. A composition according to claim 5, wherein the pigments of zinc oxide are present in the composition in an amount ranging from 5 to 55% by weight, relative to the total weight of the composition.

10. A composition according to claim 9, wherein the pigments of zinc oxide are present in the composition in an amount ranging from 15 to 25% by weight, relative to the total weight of the composition.

11. A composition according to claim 5, wherein the pigments of titanium dioxide are present in the composition in an amount ranging from 3 to 55% by weight, relative to the total weight of the composition.

12. A composition according to claim 11, wherein the pigments of titanium dioxide are present in the composition in an amount ranging from 15 to 25% by weight, relative to the total weight of the composition.

13. A composition according to claim 5, wherein the pigments of zinc oxide and the pigments of titanium dioxide are present in the composition in an amount such that the weight ratio of zinc oxide to titanium dioxide ranges from 0.5 to 2.

14. A composition according to claim 13, wherein the pigments of zinc oxide and the pigments of titanium dioxide are present in the composition in an amount such that the weight ratio of zinc oxide to titanium dioxide ranges from 0.9 to 1.7.

15. A composition according to claim 5, wherein the pigments of iron oxide and zinc oxide and the pigments of titanium dioxide are present in the composition in an amount such that the weight ratio of the sum of the zinc oxides and/or titanium dioxides to the iron oxides ranges from 0.1 to 10.

16. A composition according to claim 15, wherein the pigments of iron oxide and zinc oxide and the pigments of titanium dioxide are present in the composition in an amount such that the weight ratio of the sum of the zinc oxides and/or titanium dioxides to the iron oxides ranges from 0.2 to 8.

17. A composition according to claim 1, wherein the at least one lamellar particle is chosen from lamellar pearlescent agents, lamellar fillers and lamellar reflecting particles.

18. A composition according to claim 1, wherein the at least one pulverulent phase comprises at least two lamellar particles of different nature.

19. A composition according to claim 18, wherein one of the lamellar particles is chosen from lamellar pearlescent agents, and the other lamellar particle is chosen from lamellar fillers.

20. A composition according to claim 19, wherein the at least one pulverulent phase comprises at least one lamellar pearlescent agent and at least two lamellar fillers.

21. A composition according to claim 17, wherein the lamellar pearlescent agent is chosen from white pearlescent agents, colored pearlescent agents and pearlescent agents made of bismuth oxychloride or based on bismuth oxychloride.

22. A composition according to claim 21, wherein said white pearlescent agents are chosen from mica coated with titanium and mica coated with bismuth oxychloride.

23. A composition according to claim 21, wherein said colored pearlescent agents are chosen from mica-titanium coated with iron oxides, mica-titanium coated with ferric blue, mica-titanium coated with chromium oxide, and mica-titanium coated with an organic pigment.

24. A composition according to claim 17, wherein the lamellar pearlescent agent is bismuth oxychloride.

25. A composition according to claim 17, wherein the lamellar pearlescent agent is present in the composition in an amount ranging from 3 to 70% by weight, relative to the total weight of the composition.

26. A composition according to claim 25, wherein the lamellar pearlescent agent is present in the composition in an amount ranging from 5 to 40% by weight, relative to the total weight of the composition.

27. A composition according to claim 17, wherein the lamellar filler is chosen from talc, mica, boron nitride, kaolin, silica, and mixtures thereof.

28. A composition according to claim 27, wherein the lamellar filler is chosen from talc, mica, boron nitride, kaolin and mixtures thereof.

29. A composition according to claim 27, wherein talc is present in the composition in an amount ranging from 5 to 55% by weight, relative to the total weight of the composition.

30. A composition according to claim 29, wherein talc is present in the composition in an amount ranging from 10 to 53% by weight, relative to the total weight of the composition.

31. A composition according to claim 27, wherein boron nitride is present in the composition in an amount ranging from 3 to 10% by weight, relative to the total weight of the composition.

32. A composition according to claim 31, wherein boron nitride is present in the composition in an amount ranging from 5 to 8% by weight, relative to the total weight of the composition.

33. A composition according to claim 17, wherein the lamellar fillers are present in the composition in an amount ranging from 20 to 70% by weight, relative to the total weight of the composition.

34. A composition according to claim 33, wherein the lamellar fillers are present in the composition in an amount ranging from 30% to 35% by weight, relative to the total weight of the composition.

35. A composition according to claim 1, wherein the lamellar particles are present in the pulverulent phase in an amount ranging from 30 to 85% by weight, relative to the total weight of the composition.

36. A composition according to claim 35, wherein the lamellar particles are present in the pulverulent phase in an amount ranging from 40 to 70% by weight, relative to the total weight of the composition.

37. A composition according to claim 1, wherein the lamellar particles and the pigments are present in the composition in an amount such that the weight ratio of lamellar particles to pigments ranges from 0.5 to 5.

38. A composition according to claim 37, wherein the lamellar particles and the pigments are present in the composition in an amount such that the weight ratio of lamellar particles to pigments ranges from 0.8 to 2.5.

39. A composition according to claim 1, wherein the at least one fatty binder further comprises an oil.

40. A composition according to claim 39, wherein the oil is chosen from silicone oils, fluorinated silicones, perfluorinated oils and fatty ester oils.

41. A composition according to claim 1, wherein the at least one fatty binder further comprises a wax and/or a silicone pasty fatty substance.

42. A composition according to claim 1, wherein the at least one fatty binder further comprises a silicone resin.

43. A composition according to claim 1, wherein said composition further comprises at least one additional wax.

44. A composition according to claim 43, wherein the at least one additional wax is chosen from:
beeswax, lanolin wax, Chinese waxes; rice wax, carnauba wax, candelilla wax, ouricury wax, cork fibre wax, sugar cane wax, Japan wax and sumac wax; montan wax, microcrystalline waxes, paraffin waxes, ozokerites, ceresin wax, lignite wax, polyethylene waxes, the waxes obtained by Fisher-Tropsch synthesis, fatty acid esters and glycerides which are concrete at 40° C.,
waxes obtained by catalytic hydrogenation of animal or vegetable oils having linear or branched C₈-C₃₂ fatty chains,
silicone waxes or fluorinated waxes,
zinc laurate, magnesium stearate, magnesium myristate, zinc stearate, and
mixtures thereof.

45. A composition according to claim 43, wherein the at least one additional wax is totally or partially in powdered form.

46. A composition according to claim 44, wherein the at least one additional wax is chosen from carnauba waxes, paraffin waxes and mixtures thereof.

47. A composition according to claim 43, wherein the at least one additional wax is present in an amount ranging from 0.5 to 15% by weight, relative to the total weight of the composition.

48. A composition according to claim 47, wherein the at least one additional wax is present in an amount ranging from 0.9 to 1.2% by weight.

49. An anhydrous cosmetic composition in powdered form, comprising:
i) 1 to 10% by weight, relative to the total weight of the composition, of at least one pigment chosen from iron oxides,
ii) 20 to 50% by weight, relative to the total weight of the composition, of pigments chosen from zinc oxides, titanium oxides and a mixture thereof,
iii) 25 to 45% by weight, relative to the total weight of the composition, of at least one lamellar pearlescent agent,
iv) 15 to 35% by weight, relative to the total weight of the composition, of at least one lamellar filler,
v) 2 to 7% by weight, relative to the total weight of the composition, of at least one fatty binder.

50. A method for making up the skin of the face and/or of the body, comprising applying to said skin an anhydrous cosmetic composition in powdered form comprising at least one pulverulent phase and at least one fatty binder, said pulverulent phase comprising:
i) 15 to 70% by weight, relative to the total weight of the composition, of at least one pigment, and,
ii) 30 to 85% by weight, relative to the total weight of the composition, of at least one lamellar particle other than the pigment.

51. A method for concealing the color defects of the skin, while conferring a natural and satiny appearance on the made-up skin, comprising applying to the skin an anhydrous cosmetic composition in powdered form comprising at least one pulverulent phase and at least one fatty binder, said pulverulent phase comprising:
i) 15 to 70% by weight, relative to the total weight of the composition, of at least one pigment, and,
ii) 30 to 85% by weight, relative to the total weight of the composition, of at least one lamellar particle other than the pigment.
